(19) United States
(12) Patent Application Publication
(10) Pub. No.: US 2026/0248835 A1
(43) Pub. Date: Aug. 27, 2026

(54) COMPOSITION FOR IMPROVED MITOCHONDRIAL FUNCTION AND CELLULAR LONGEVITY

(71) Applicant: Spartacus Brands, LLC, Boca Raton, FL (US)

(72) Inventors: Augustin Quancard, Boca Raton, FL (US); Gregory Papigny, Boca Raton, FL (US); Jeremy Eisenberg, Boca Raton, FL (US); Stephanie Eisenberg, Boca Raton, FL (US); Gary Bitton, Boca Raton, FL (US)

(21) Appl. No.: 19/061,548

(22) Filed: Feb. 24, 2025

(51) Int. Cl. A61K 31/7084; A61K 9/00; A61K 31/05; A61K 31/12; A61K 31/164; A61K 31/221; A61K 31/37; A61K 31/4745; A61K 31/706; A61K 47/24; A61K 47/38

(52) U.S. Cl. CPC A61K 31/7084; A61K 9/0053; A61K 31/05; A61K 31/12; A61K 31/164; A61K 31/221; A61K 31/37; A61K 31/4745; A61K 31/706; A61K 47/24; A61K 47/38

(57) ABSTRACT

The present invention discloses a water soluble composition for oral consumption including nicotinamide adenine dinucleotide (NAD+) and urolithin. The composition provides a synergistic combination of NAD+ and urolithin, offering various health benefits. The inclusion of NAD+ and urolithin in a single formulation offers a convenient and effective means for oral consumption, making it suitable for a wide range of applications in the field of nutrition and wellness.

### GOVERNMENT CONTRACT

Not applicable.

### CROSS REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The present disclosure generally relates to supplemental nutrition and more particularly, to supplemental nutrition for improved mitochondrial function and cellular longevity.

### BACKGROUND

Ellagitannins are a class of antioxidant polyphenols found in many fruits, berries and nuts. During juice processing, high levels end up extracted from fruit peels into the final juice product. In vitro studies show ellagitannins have potential health benefits at nutritional concentrations. For instance, pomegranate juice owes its potent antioxidant activity mainly to punicalagin ellagitannins.

However, ellagitannins themselves have low gut absorption. But certain metabolites produced from them by intestinal bacteria can get absorbed. Ellagitannins are converted to ellagic acid in the gut, which microflora further metabolize into derivatives called urolithins that enter circulation.

Research links mitochondrial performance and reactive oxygen species production to aging. Declining mitochondrial energy output and excess oxidative stress play a key role in neurodegeneration, metabolic disorders and the overall aging process. Neuronal cells are especially vulnerable to imbalanced redox states and energy deficits-manifesting as cognitive impairment.

Maintaining mitochondrial fitness is thus tied to preventing oxidation-related inflammation and cell death pathways that drive disease progression. Bioactive foods like ellagitannin-rich fruits may aid in supporting mitochondrial homeostasis. Their derived metabolites and effects on cellular signaling present therapeutic potential to counteract metabolic, inflammatory and neurologic conditions involving mitochondrial distress.

Rinsch et. al. discloses compositions containing urolithin compounds or precursors, which can be used for treating or preventing diseases related to insufficient mitochondrial activity, including aging, stress, diabetes, obesity, and neurodegenerative disorders. Rinsch specifically focused on ellagitannins and urolithin A and disclosed such compounds being formulated as foods or dietary supplements. In addition to disease treatment, the urolithin compositions disclosed by Rinsch can beneficially be used in healthy individuals as well, in order to increase or maintain metabolic rate, decrease percentage of body fat, increase or preserve muscle mass, manage body weight, improve or sustain mental ability and memory, enhance muscle performance, improve or sustain positive mood, and manage stress. However, Rinsch merely disclosed the urolithin as a standalone compound with only trace amounts of other ingredients.

The effect of urolithin on mitochondrial function is profound. It activates mitophagy, the process by which damaged mitochondria are selectively degraded and recycled. This activity is crucial for maintaining mitochondrial quality and function, ensuring that cells have a healthy powerhouse to generate energy efficiently. Improved mitochondrial function, in turn, supports better energy metabolism in cells, which is fundamental for overall health and vitality. In terms of cellular longevity, urolithin's role in enhancing mitophagy directly contributes to the longevity of cells. By removing damaged mitochondria, urolithin helps to prevent the accumulation of mitochondrial dysfunction, a key factor in cellular aging. This action can lead to an extension of the healthy lifespan of cells, potentially slowing down the aging process and mitigating age-related decline in cellular function.

Although various solutions have been proposed to improve mitochondrial function, none combines the characteristics of the present invention. Thus, there remains a need to increase the mitochondrial functionality and enhance the cellular longevity simultaneously.

### SUMMARY

This invention disclosure pertains to a nutritional composition containing two key bioactive components—nicotinamide adenine dinucleotide (NAD+) and urolithin—that is aimed at achieving synergistic improvements in mitochondrial health and cellular longevity through their combination.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

According to an embodiment, the composition described herein relates to a water soluble composition for oral consumption including nicotinamide adenine dinucleotide (NAD+) and urolithin.

In some embodiments, the composition described herein relates to a composition which further includes nicotinamide mononucleotide (NMN).

According to another embodiment, the composition described herein relates to an ingestible composition for improved mitochondrial function, including nicotinamide adenine dinucleotide (NAD+), and urolithin.

In some embodiments, the composition described herein relate to a composition for improved mitochondrial function, in which the urolithin is a urolithin A.

In some embodiments, the composition described herein relates to a composition for improved mitochondrial function, in which the composition further includes a phospholipid complex from sunflower and lecithin.

In some embodiments, the composition described herein relates to a composition for improved mitochondrial function, in which the composition further includes palmitoylethanolamide (PEA).

In some embodiments, the composition described herein relates to a composition for improved mitochondrial function, in which the composition further includes acetyl-L-carnitine.

In some embodiments, the composition described herein relates to a composition for improved mitochondrial function, in which the composition further includes trans-resveratrol.

In some embodiments, the composition described herein relates to a composition for improved mitochondrial function, in which the composition further includes curcumin.

In some embodiments, the composition described herein relates to a composition for improved mitochondrial function, in which the composition further includes pyrroloquinoline quinone (PQQ).

According to yet another embodiment, the composition described herein relates to a composition for improved mitochondrial function, in which the composition further includes microcrystalline cellulose.

In some embodiments, the composition described herein relates to a composition for improved mitochondrial function, in which the composition further includes nicotinamide mononucleotide (NMN).

In some embodiments, the composition described herein relate to an ingestible composition for improved cellular longevity, including nicotinamide adenine dinucleotide (NAD+), and urolithin.

In some embodiments, the composition described herein relates to a composition for improved cellular longevity further includes nicotinamide mononucleotide (NMN), a phospholipid complex from sunflower and lecithin, and palmitoylethanolamide (PEA).

In some embodiments, the composition described herein relates to a composition for improved cellular longevity, in which the urolithin is a urolithin A.

In some embodiments, the composition described herein relates to a composition for improved cellular longevity, in which the composition further includes acetyl-L-carnitine.

In some embodiments, the composition described herein relates to a composition for improved cellular longevity, in which the composition further includes trans-resveratrol.

In some embodiments, the composition described herein relates to a composition for improved cellular longevity, in which the composition further includes curcumin.

In some embodiments, the composition described herein relates to a composition for improved cellular longevity, in which the composition further includes pyrroloquinoline quinone (PQQ).

In some embodiments, the composition described herein relates to a composition for improved cellular longevity, in which the composition further includes microcrystalline cellulose.

It is contemplated that disclosed composition and claims provided herein may have the following advantages:
a) Improved mitochondrial function
b) Enhanced cellular longevity
c) Improved muscle health
d) Improved gut health
e) Neuroprotection

One or more of the above-disclosed embodiments, in addition to certain alternatives, are provided in further detail below. The disclosed subject matter is not, however, limited to any particular embodiment disclosed.

### DETAILED DESCRIPTION

According to an embodiment, the composition described herein relates to a water soluble composition for oral consumption including nicotinamide adenine dinucleotide (NAD+) and urolithin. NAD+ is an essential coenzyme and cofactor that enables numerous redox reactions central to fuel catabolism processes and energy production pathways in cells. NAD+ gets continually recycled between its oxidized and reduced forms to accept and donate electrons during carbon breakdown stages like glycolysis and the Kreb's cycle, ultimately generating ATP molecules used for cellular work. Hence, NAD+'s integral role in modulating mitochondrial performance and metabolism has been long recognized. In several embodiments, the composition can be an ingestible composition for improved cellular longevity and/or improved mitochondrial function.

In some embodiments, oral doses of NAD+ can stimulate mitochondrial biogenesis, curb inflammatory drivers of aging, and stimulate sirtuins and other longevity pathways.

In some embodiments, urolithin or its precursors such as ellagitannins, which can be found in fruits and nuts, are used as a mitochondrial supporter. Urolithins can be utilized to enhance mitochondrial membrane potential, increase oxidative capacity, improve fitness and mitigate damage through coordinated mTOR, sirtuin and mitophagy pathways.

In some embodiments, the composition further includes nicotinamide mononucleotide (NMN). In an embodiment, NMN which is a NAD+ precursor can replenish declining NAD+ availability in cells to enhance mitochondrial fitness and sustain efficient energy metabolism. Further, oral supplementation can counteract age-related NAD+ reductions. In an embodiment, NMN effectively crosses cell membranes via specialized transporters and directly converts to NAD+ through enzymatic phosphorylation to activate sirtuins, maintain redox capacity, and support electron transport chain activity driving ATP regeneration. By stimulating sirtuin enzymes, NMN administration can induce mitochondrial biogenesis—generating new mitochondria to meet cellular energy demands. In an embodiment, the increased mitochondrial network protects against mutations in existing mitochondria. As a nucleotide, NMN also gets incorporated into NAD+ biosynthesis pathways from scratch, providing an alternative supply to stimulate greater coenzyme generation endogenously beyond recycling pathways. In an embodiment, NMN exhibits additional specialized signaling functions via activation of NAD+-consuming enzymes called CD38 and PARPs involved in DNA repair and gene expression regulation that regulate health span.

In some embodiments, the urolithin is urolithin A, which can activate mitophagy (the selective degradation of mitochondria) helping to maintain cellular health by removing damaged or dysfunctional mitochondria. In some embodiments, urolithin A can prevent cellular damage and aging. Urolithin A also exhibits anti-inflammatory effects by inhibiting the activity of NF-kB, a protein complex that plays a role in inflammatory responses. Additionally, in an embodiment, urolithin A's antioxidant properties help to neutralize free radicals, reducing oxidative stress which is linked to the development of chronic diseases and aging.

In some embodiments, urolithin A has a positive impact on aging and longevity by enhancing mitochondrial function, slowing down the aging process, and improving muscle function and endurance. In an embodiment, urolithin A causes neuroprotective effects, where its anti-inflammatory and antioxidant activities protect against neurodegenerative diseases. In additional embodiments, urolithin A can help in modulating lipid and glucose metabolism, which is beneficial for conditions such as obesity and diabetes. Furthermore, in another embodiment, urolithin A has anticancer properties through mechanisms such as apoptosis induction and the inhibition of cancer cell growth.

In some embodiments, the composition includes phospholipid complex. The phospholipid complex can be derived from sunflower and lecithin. Such sunflower-derived phospholipids can be extracted from sunflower seeds and can be non-soy, non-GMO sources of lecithin. In an embodiment, lecithin can be substituted by another fatty substance that occurs in animal and plant tissues. In some embodiments, a phosphatidylcholine-rich, phosphatidylinositol-rich, and phosphatidylethanolamine-rich sunflower lecithin is used. Phosphatidylcholine, phosphatidylinositol, and phosphatidylethanolamine are all types of phospholipids essential for the structural integrity of cell membranes. As a non-limiting example, phosphatidylcholine can be used, which is a major component of cell membranes and is involved in metabolism and liver function. These phospholipids also play a role in the body's lipid transport system, aiding in the digestion and movement of fats.

In some embodiments, the composition includes palmitoylethanolamide (PEA), which is a fatty acid amide belonging to a class of naturally occurring lipid mediators in the body. In some embodiments, PEA enhances the activity of endocannabinoids and acts on several targets within cells, contributing to its anti-inflammatory and analgesic effects.

In another embodiment, PEA modulates the body's pain and inflammatory responses through a mechanism known as the entourage effect, where PEA enhances the effects of other endocannabinoids produced by the body to reduce pain and inflammation. In additional embodiments, PEA interacts with peroxisome proliferator-activated receptors (PPARs), especially PPAR-alpha, to exert its anti-inflammatory actions. By activating these receptors, PEA helps to reduce the expression of genes involved in inflammation, thus decreasing the production of proinflammatory molecules.

In some embodiments, PEA can facilitate treating chronic pain conditions, such as neuropathic pain, fibromyalgia, and osteoarthritis, and reduce pain intensity for sufferers. In some embodiments, PEA can help manage conditions characterized by chronic inflammation, including certain types of chronic pain, inflammatory bowel disease, and even neuroinflammatory conditions.

In some embodiments, the composition includes acetyl-L-carnitine, which is a form of the amino acid L-carnitine. Acetyl-L-carnitine can facilitate transporting fatty acids into the mitochondria, where fatty acids are converted into energy. This process is especially important for the heart and muscle function, as well as for a range of bodily processes that require a significant amount of energy. In some embodiments, acetyl-L-carnitine has neuroprotective properties and can support brain health in various ways. Acetyl-L-carnitine can cross the blood-brain barrier, a unique property that allows it to directly influence brain function. Once in the brain, acetyl-L-carnitine contributes to the production of acetylcholine, a neurotransmitter essential for memory and learning processes. In some embodiments, acetyl-L-carnitine can help improve cognitive function, enhance memory, and slow down the progression of age-related cognitive decline.

In some embodiments, acetyl-L-carnitine combats oxidative stress, and helps protect cells from damage caused by free radicals. This antioxidant property can be beneficial not only for brain health but also for preventing damage to other tissues and organs in the body. In an embodiment, acetyl-L-carnitine can help reduce pain and improve nerve function in people with conditions such as diabetic neuropathy and support cardiovascular health by improving blood flow and reducing symptoms of heart-related conditions.

In some embodiments, where the consumer faces fatigue or in case the consumer is looking to enhance their physical performance, acetyl-L-carnitine can offer an energy boost, making it easier to sustain higher levels of physical activity. Acetyl-L-carnitine role in fat metabolism can also support weight management efforts by enhancing the body's ability to burn fat.

In some embodiments, the composition includes trans-resveratrol, which is a naturally occurring compound found in various plants, including the skin of red grapes, berries, and peanuts. Trans-resveratrol is a form of resveratrol (a polyphenolic compound known for its antioxidant properties) and the “trans” prefix denotes its specific chemical structure, which is thought to be more biologically active and beneficial than its “cis” counterpart. The functionality of trans-resveratrol is primarily linked to its potent antioxidant and anti-inflammatory properties. In some embodiments, by scavenging free radicals and inhibiting inflammation, trans-resveratrol helps protect cells and tissues from oxidative stress and damage, which are implicated in a wide range of chronic diseases. This action is crucial for maintaining cellular health and preventing the onset of conditions associated with aging and environmental stressors.

In some embodiments, trans-resveratrol can further activate sirtuins, particularly SIRT1, a group of enzymes that play key roles in cellular regulation, including aging, metabolism, and stress resistance. In various embodiments, activation of SIRT1 by trans-resveratrol can mimic the effects of caloric restriction, a known factor in extending lifespan and enhancing health span. In some embodiments, such an activation leads to improved mitochondrial function, enhanced energy production, and slower aging processes.

In some embodiments, trans-resveratrol contributes to heart health by improving endothelial function, which is crucial for blood vessel flexibility and blood pressure regulation. In Further embodiments, trans-resveratrol helps to reduce LDL cholesterol oxidation and platelet aggregation, lowering the risk of atherosclerosis and heart disease. In an embodiment, trans-resveratrol can improve the cancer prevention by influencing various stages of cancer development, including initiation, promotion, and progression, by modulating inflammation, cell proliferation, and apoptosis. Trans-resveratrol can improve insulin sensitivity and glucose metabolism. This action is particularly relevant in the context of diabetes and metabolic syndrome, where improved insulin function can significantly impact health outcomes. In another embodiment, trans-resveratrol ha neuroprotective effects to protect brain cells from damage and support cognitive function.

In some embodiments, the composition includes curcumin. Curcumin's functionality and benefits stem from its potent anti-inflammatory and antioxidant properties. In several embodiments, by blocking NF-kB, a molecule that travels into the nuclei of cells and turns on genes related to inflammation, curcumin acts at the biochemical level to fight inflammation. In an embodiment, curcumin acts as an antioxidant that can neutralize free radicals due to its chemical structure. Furthermore, curcumin boosts the activity of the body's own antioxidant enzymes, thus providing a double barrier against oxidative stress, which plays a role in many diseases and has been linked to the aging process.

Curcumin improves the function of the endothelium, the lining of the blood vessels, which regulates blood pressure, blood clotting, and various other factors critical to heart health. Curcumin can cross the blood-brain barrier and act as an agent in the prevention and treatment of Alzheimer's disease. In some embodiments, for cancer prevention and treatment, curcumin affects cancer growth, development, and spread at the molecular level. Curcumin can reduce angiogenesis (growth of new blood vessels in tumors), metastasis (spread of cancer), and contribute to the death of cancerous cells. Curcumin can facilitate treating symptoms of arthritis, outperforming anti-inflammatory drugs without the side effects. Curcumin can further boost brain neurotransmitters serotonin and dopamine.

In some embodiments, the composition includes pyrroloquinoline quinone (PQQ). PQQ is a small quinone molecule found in small amounts in various foods and is considered a novel cofactor with antioxidant and B-vitamin-like activity. In some embodiments, PQQ facilitates the growth and development of new mitochondria, the energy-generating compartments in cells, through a process known as mitochondrial biogenesis. This is crucial for maintaining optimal energy levels in the body and supporting the health and function of various tissues and organs. In various embodiments, by increasing the number and efficiency of mitochondria, PQQ can enhance energy production, which is vital for physical performance, recovery, and overall vitality.

In several embodiments, PQQ is capable of scavenging reactive oxygen species (ROS), reducing oxidative stress, and thereby protecting cells from damage. This antioxidant action is more potent than that of many other antioxidants, making PQQ highly effective in combating oxidative stress-related damage, which is implicated in aging and many chronic diseases.

In an embodiment, PQQ can support nerve growth factor (NGF) production and stimulate the growth of nerve cells, thereby improving memory and cognitive functions. In such an embodiment, PQQ can facilitate preventing and managing neurodegenerative diseases such as Alzheimer's disease and in enhancing brain health more generally.

In some embodiments, PQQ can help reduce the size of damaged areas caused by acute heart attacks. In an embodiment, PQQ can influence pathways involved in insulin sensitivity and glucose metabolism. PQQ can support immune function through its antioxidant activity and modulation of signaling pathways, which can reduce inflammation and enhancing the body's resistance to infections and diseases.

It should be noted that other ingredients such as turmeric extract (e.g., curcuma longa), pomegranate fruit extract, green tea extract, coenzyme Q10 (C0Q10), Microcrystalline Cellulose, or any other suitable ingredients can be added to the disclosed composition.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSION, RAMIFICATION, AND SCOPE

While certain embodiments of the invention have been illustrated and described, various modifications are contemplated and can be made without departing from the spirit and scope of the invention. For example, the composition may comprise any active ingredient, including pharmaceuticals, vitamins, and other supplements. Accordingly, it is intended that the invention is not limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers are incorporated herein by reference. Aspects of the invention can be modified, if necessary, to employ the systems, functions and concepts of the various references described above to provide yet further embodiment of the invention.

Particular terminology used when describing certain features or aspects of the invention should not be taken to imply that the terminology is being refined herein to be restricted to any specific characteristics, features, or aspects of the composition for liposomal delivery of supplemental creatine with which that terminology is associated. In general, the terms used in the following claims should not be constructed to limit the composition for supplemental creatine to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the composition for supplemental creatine is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the composition for supplemental creation.

What is claimed is:

1. A water soluble composition for oral consumption comprising:
nicotinamide adenine dinucleotide (NAD+); and
urolithin.

2. The composition of claim 1, further comprising Nicotinamide mononucleotide (NMN).

3. An ingestible composition for improved mitochondrial function, comprising:
nicotinamide adenine dinucleotide (NAD+); and
urolithin.

4. The composition of claim 3, wherein the urolithin is a urolithin A.

5. The composition of claim 3, wherein the composition further comprises a phospholipid complex from sunflower and lecithin.

6. The composition of claim 3, wherein the composition further comprises palmitoylethanolamide (PEA).

7. The composition of claim 3, wherein the composition further comprises acetyl-L-carnitine.

8. The composition of claim 3, wherein the composition further comprises trans-resveratrol.

9. The composition of claim 3, wherein the composition further comprises curcumin.

10. The composition of claim 3, wherein the composition further comprises pyrroloquinoline quinone (PQQ).

11. The composition of claim 3, wherein the composition further comprises microcrystalline cellulose.

12. The composition of claim 3, wherein the composition further comprises nicotinamide mononucleotide (NMN).

13. An ingestible composition for improved cellular longevity, comprising:
nicotinamide adenine dinucleotide (NAD+); and
urolithin.

14. The composition of claim 13, further comprising:
nicotinamide mononucleotide (NMN);
a phospholipid complex from sunflower and lecithin; and
palmitoylethanolamide (PEA).

15. The composition of claim 13, wherein the urolithin is a urolithin A.

16. The composition of claim 13, wherein the composition further comprises acetyl-L-carnitine.

17. The composition of claim 13, wherein the composition further comprises trans-resveratrol.

18. The composition of claim 13, wherein the composition further comprises curcumin.

19. The composition of claim 13, wherein the composition further comprises pyrroloquinoline quinone (PQQ).

20. The composition of claim 13, wherein the composition further comprises microcrystalline cellulose.
